(19) United States
(12) Patent Application Publication
(10) Pub. No.: US 2026/0356854 A1
(43) Pub. Date: Nov. 12, 2026

(54) Prodrugs of Varenicline and Compounds Containing Vulnerable Amines

(71) Applicant: Pfizer Inc., New York, NY (US)

(72) Inventors: Kapildev Kashmirilal Arora, East Lyme, CT (US); Macy Michele Burachio, Groton, CT (US); Zhengong Bryan Li, Escondido, CA (US); Ronald Scott Obach, Groton, CT (US)

(73) Assignee: Pfizer Inc., New York, NY (US)

(21) Appl. No.: 19/125,369

(22) PCT Filed: Nov. 1, 2023
(86) PCT No.: PCT/IB2023/060997

§ 371 Date: Apr. 29, 2025

Related U.S. Application Data
(60) Provisional application No. 63/382,464, filed on Nov. 4, 2022.

(51) Int. Cl. C07D 519/00; A61K 31/55; A61P 25/34

(52) U.S. Cl. CPC C07D 519/00; A61K 31/55; A61P 25/34

(57) ABSTRACT

The present invention relates to prodrugs of compounds that contain vulnerable amines (e,g, secondary, tertiary and quaternary amines), and methods for their manufacture.

### FIELD OF THE INVENTION

The present invention relates to prodrugs of compounds that contain vulnerable amines (e,g, secondary, tertiary and quaternary amines) susceptible to form nitrosamines, and methods for their manufacture. The use of the prodrugs described herein help preventing/reducing the level of nitrosamines agents that may occur with pharmaceutical active ingredients (APIs) containing vulnerable amines in a drug product.

### BACKGROUND OF THE INVENTION

Substances that react with DNA have the potential to cause mutations that may cause cancer. New drugs and pharmaceutical excipients are accordingly screened in a bacterial mutagenicity assay at a preclinical stage of drug development in order to identify any potential toxicity of this kind. Some functional groups found in organic compounds are known from their structure alone to be highly potent mutagens and their formation must be strictly controlled in order to minimize carcinogenic risk. The N-nitroso functional group, present in nitrosamines and nitrosamides, is one such group of concern. A very large proportion of compounds comprising this functional group, when activated metabolically, alkylate DNA, leading to mutation and cancer. In humans, nitrosamines tend to be the most common N-nitroso compounds observed and have been linked to cancers such as kidney, liver and lung cancer.

One source of nitrosamines in humans is the reaction of activated nitrite with amines, particularly secondary amines, which are present in a wide range of biological molecules and drugs. The need to minimize the exposure of humans to N-nitroso compounds, and therefore to nitrite, has been increasingly recognized in recent years and the level of these compounds in marketed drugs and drugs undergoing regulatory approval has been subject to increasing levels of scrutiny. Other nitrosating agents such as nitrite esters can also be present. Many common excipients used in the formulation of drugs contain ppm levels of nitrite and other nitrosating agents and consequently have the potential to lead to the formation nitrosamines in the drug product in excess of the required acceptable daily intake level.

Therefore, a need exists to provide drug products that are nitrosamine free or drug products that contain acceptable daily intake levels of nitrite and other nitrosating agents. The present invention provides a solution to this problem utilizing a prodrug approach such that the prodrugs described herein have the advantage of providing a drug product that remains nitrosamine free over time. It has indeed been found that the replacement in a drug product of the API that contain one or more vulnerable amines (i.e., secondary, tertiary and quaternary amines) susceptible to form nitrosamines by a prodrug wherein the one or more vulnerable amine is/are derivatized allows obtaining bioequivalent drug products which are free of nitrosamine impurities.

### SUMMARY OF THE INVENTION

The present invention is directed to a compound that is (R)-6-methyl-1-((6R,10S)-6,7,9,10-tetrahydro-8H-6,10-methanoazepino[4,5-g]quinoxalin-8-yl)-1,3-dihydrofuro[3,4-c]pyridin-7-ol, or a pharmaceutically acceptable salt thereof, tautomer thereof, or pharmaceutically acceptable salt of a tautomer thereof.

The present invention is also directed to a composition comprising a prodrug of a compound having a secondary, tertiary, or quaternary amine and at least one pharmaceutically acceptable excipient, wherein the composition is free of nitrosamine impurities.

The present invention is also directed to a composition comprising a prodrug of a compound having a secondary, tertiary, or quaternary amine and at least one pharmaceutically acceptable excipient, wherein the composition has less than 0.03 ppm nitrosamine impurities.

The present invention is also directed to use of a prodrug in a drug product in replacement of a drug in an original composition having a level of nitrosamine impurities above 0.03 ppm, wherein the drug product comprising said prodrug is bioequivalent to the original composition and is free of nitrosamine impurities.

The present invention is also directed to a use of a prodrug in a drug product, wherein the drug product comprises a compound having a secondary, tertiary or quaternary amine, and wherein the drug product has less than 0.03 ppm nitrosamine impurities.

### BRIEF DESCRIPTION OF THE DRAWINGS

FIG. 1 is a graph that shows the Powder X-ray diffraction pattern of Compound 1.

FIG. 2 is an asymmetric unit diagram with anisotropic displacement parameters drawn at 50% probability for Compound 1.

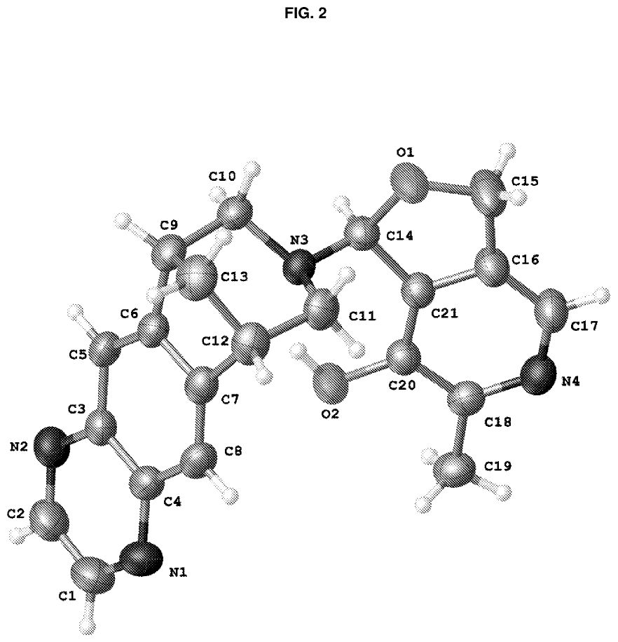

FIG. 3 is a graph of the mean monkey plasma concentrations of varenicline following an oral dose of 0.04 mg eq/kg of prodrug (Compound 1) and varenicline tartrate.

FIG. 4 is a schematic of three tautomers of varenicline prodrug and the release of varenicline API via chemical hydrolysis.

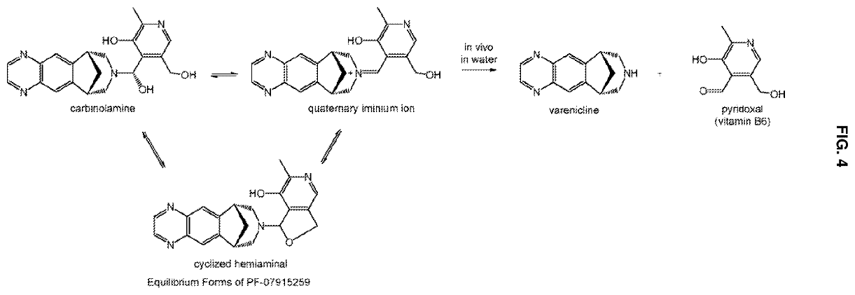

### DETAILED DESCRIPTION OF THE INVENTION

The present invention may be understood more readily by reference to the following detailed description of exemplary embodiments of the invention and the examples included therein.

It is to be understood that the terminology used herein is for the purpose of describing particular embodiments only and is not intended to be limiting. In this specification and in the claims that follow, reference will be made to a number of terms that shall be defined to have the following meanings:

As used herein in the specification, “a” or “an” may mean one or more. As used herein in the claim(s), when used in conjunction with the word “comprising”, the words “a” or “an” may mean one or more than one. As used herein “another” may mean at least a second or more.

“Compounds” when used herein includes any pharmaceutically acceptable derivative or variation, including conformational isomers (e.g., cis and trans isomers) and all optical isomers (e.g., enantiomers and diastereomers), racemic, diastereomeric and other mixtures of such isomers, as well as solvates, hydrates, isomorphs, polymorphs, tautomers, esters, salt forms, and prodrugs. The expression “prodrug” refers to compounds that are drug precursors which following administration, release the drug in vivo via some chemical or physiological process (e.g., a prodrug on being brought to the physiological pH or through enzyme action is converted to the desired drug form).

The term “nitrosamine describes a class of compounds having the chemical structure of a nitroso group bonded to an amine (R1N(—R2)-N═O). The compounds can form by a nitrosating reaction between amines (secondary, tertiary, or quaternary amines) and nitrous acid (nitrite salts under acidic conditions). Examples of nitrosamines include, but are not limited to NDMA, N-nitrosodiethylamine (NDEA), N-nitroso-N-methyl-4-aminobutanoic acid (NMBA), N-nitrosoisopropylethyl amine (NIPEA), N-nitrosodiisopropylamine (NDIPA), N-nitrosodibutylamine (NDBA), and N-nitrosomethylphenylamine (NMPA).

The term “free of nitrosamine” or “nitrosamine free” means that there is no detectable level of nitrosamine impurities in the drug product.

“Patient” refers to warm blooded animals such as, for example, guinea pigs, mice, rats, gerbils, cats, rabbits, dogs, cattle, goats, sheep, horses, monkeys, chimpanzees, and humans.

The term “pharmaceutically acceptable” means the substance (e.g., the compounds of the invention) and any salt thereof, or composition containing the substance or salt of the invention that is suitable for administration to a patient.

“Therapeutically effective amount” means an amount of a compound of the present invention that (i) treats or prevents the particular disease, condition, or disorder, (ii) attenuates, ameliorates, or eliminates one or more symptoms of the particular disease, condition, or disorder, or (iii) prevents or delays the onset of one or more symptoms of the particular disease, condition, or disorder described herein.

The term “treating”, “treat” or “treatment” as used herein embraces both preventative, i.e., prophylactic, and palliative treatment, i.e., relieve, alleviate, or slow the progression of the patient's disease (or condition) or any tissue damage associated with the disease.

The presence of unacceptable levels of nitrosamine impurities in pharmaceutical products has been investigated by various regulatory agencies for a number of years leading to coordinated partnerships for communicating analytical methods to detect and identify various nitrosamines, and to develop rapid solutions to ensure the safety and quality of the drug supply. The U.S. FDA has identified seven nitrosamine impurities that could be present in drug products: NDMA, N-nitrosodiethylamine (NDEA), N-nitroso-N-methyl-4-aminobutanoic acid (NMBA), N-nitrosoisopropylethyl amine (NIPEA), N-nitrosodiisopropylamine (NDIPA), N-nitrosodibutylamine (NDBA), and N-nitrosomethylphenylamine (NMPA). Five of them (NDMA, NDEA, NMBA, NIPEA, and NMPA) have actually been detected in drug substances or drug products. Some of these impurities have been classified as potent genotoxic agents in several animal species and some are classified as probable or possible human carcinogens by the International Agency for Research on Cancer (IARC). As a result, the FDA has published interim acceptable limits for these impurities.

The embodiments described below in connection with the present invention are a solution to the nitrosamine in drug product issue.

In a first embodiment E1, the invention provides a compound (Compound 1) of the formula:

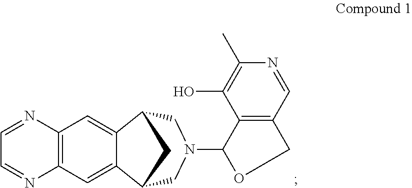 [SMILES: Cc1ncc2c(c1O)C(N1C[C@H]3C[C@@H](C1)c1cc4nccnc4cc13)OC2]

or a pharmaceutically acceptable salt thereof, tautomer thereof, or pharmaceutically acceptable salt of said tautomer thereof.

In a further embodiment E2, the invention of embodiment E1 the compound (Compound 1) is a crystalline form.

In a further embodiment E3, the invention of embodiment E1 the crystalline form of Compound 1 exhibits a powder X-ray diffraction pattern (PXRD) having at least one characteristic peak expressed in degrees 2θ (CuKα radiation) selected from the group consisting of 10.4±0.2° 2θ, 11.3±0.2° 2θ, 13.5±0.2° 2θ, 14.2±0.2° 2θ, 17.9±0.2° 2θ, and 24.8±0.2° 2θ.

In a further embodiment E4, the invention of embodiment E1 the crystalline form exhibits a powder X-ray diffraction pattern (PXRD) having at least two characteristic peak expressed in degrees 2θ (CuKα radiation) selected from the group consisting of 10.4±0.2° 2θ, 11.3±0.2° 2θ, 13.5±0.2° 2θ, 14.2±0.2° 2θ, 17.9±0.2° 2θ, and 24.8±0.2° 2θ.

In a further embodiment E5, the invention of embodiment E1 the crystalline form exhibits a powder X-ray diffraction pattern (PXRD) having at least three characteristic peak expressed in degrees 2θ (CuKα radiation) selected from the group consisting of 10.4±0.2° 2θ, 11.3±0.2° 2θ, 13.5±0.2° 2θ, 14.2±0.2° 2θ, 17.9±0.2° 2θ, and 24.8±0.2° 2θ.

In a further embodiment E6, the invention of embodiment E1 the crystalline form exhibits a powder X-ray diffraction pattern (PXRD) having at least four characteristic peak expressed in degrees 2θ (CuKα radiation) selected from the group consisting of 10.4±0.2° 2θ, 11.3±0.2° 2θ, 13.5±0.2° 2θ, 14.2±0.2° 2θ, 17.9±0.2° 2θ, and 24.8±0.2° 2θ.

In a further embodiment E7, the invention of embodiment E1 the crystalline form exhibits a powder X-ray diffraction pattern (PXRD) having at least five characteristic peak expressed in degrees 2θ (CuKα radiation) selected from the group consisting of 10.4±0.2° 2θ, 11.3±0.2° 2θ, 13.5±0.2° 2θ, 14.2±0.2° 2θ, 17.9±0.2° 2θ, and 24.8±0.2° 2θ.

In a further embodiment E8, the invention of embodiment E1 the crystalline form exhibits a powder X-ray diffraction pattern (PXRD) having characteristic peaks expressed in degrees 2θ (CuKα radiation) from 10.4±0.2° 2θ, 11.3±0.2° 2θ, and 13.5±0.2° 2θ.

In a further embodiment E9, the invention of embodiment E1 the crystalline form exhibits a powder X-ray diffraction pattern (PXRD) having characteristic peaks expressed in degrees 2θ (CuKα radiation) from 10.4±0.2° 2θ, 11.3±0.2° 2θ, 13.5±0.2° 2θ, and 14.2±0.2° 2θ.

In a further embodiment E10, the invention of embodiment E1 the crystalline form exhibits a powder X-ray diffraction pattern (PXRD) having characteristic peaks expressed in degrees 2θ (CuKα radiation) from 10.4±0.2° 2θ, 11.3±0.2° 2θ, 13.5±0.2° 2θ, 14.2±0.2° 2θ, and 17.9±0.2° 2θ.

In a further embodiment E11, the invention of embodiment E1 the crystalline form exhibits a powder X-ray diffraction pattern (PXRD) having characteristic peaks expressed in degrees 2θ (CuKα radiation) from 10.4±0.2° 2θ, 11.3±0.2° 2θ, 13.5±0.2° 2θ, 14.2±0.2° 2θ, 17.9±0.2° 2θ, and 24.8±0.2° 2θ.

In a further embodiment E12, the invention provides a compound (Compound 2) of the formula:

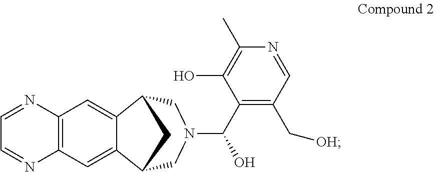 [SMILES: Cc1ncc(CO)c([C@H](O)N2C[C@H]3C[C@@H](C2)c2cc4nccnc4cc23)c1O]

or a pharmaceutically acceptable salt thereof, tautomer thereof, or pharmaceutically acceptable salt of said tautomer thereof.

In a further embodiment of E12, the invention provides a compound (Compound 2a) of the formula:

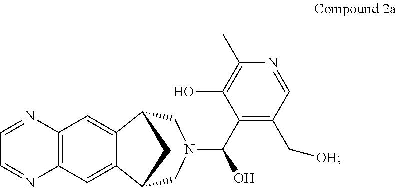 [SMILES: Cc1ncc(CO)c([C@@H](O)N2C[C@H]3C[C@@H](C2)c2cc4nccnc4cc23)c1O]

or a pharmaceutically acceptable salt thereof, tautomer thereof, or pharmaceutically acceptable salt of said tautomer thereof.

In a further embodiment E13, the invention provides a compound (Compound 2) of the formula:

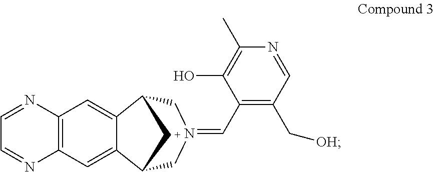

or a pharmaceutically acceptable salt thereof, tautomer thereof, or pharmaceutically acceptable salt of said tautomer thereof.

In a further embodiment E14, the invention provides a composition containing a compound according to any one of embodiments E1 to E13, and at least one pharmaceutically acceptable excipient, wherein the composition is free of nitrosamine impurities.

In an further embodiment E15, the invention provides a composition containing a compound according to any one of embodiments E1 to E13, and at least one pharmaceutically acceptable excipient, wherein the composition has less than 0.03 ppm nitrosamine impurities.

In a further embodiment of E16, the composition has less than 0.003 ppm nitrosamine impurities.

In a further embodiment E17, the invention provides a composition comprising a prodrug of a compound having a secondary, tertiary, or quaternary amine and at least one pharmaceutically acceptable excipient, wherein the composition is free of nitrosamine impurities.

In a further embodiment of E17, the composition has less than 0.03 ppm nitrosamine impurities.

In yet another embodiment of E17, the composition has less than 0.003 ppm nitrosamine impurities.

In a further embodiment (E18), the invention provides a use of a prodrug in a drug product, wherein the drug product comprises a compound having a secondary, tertiary or quaternary amine, and wherein the drug product is free of nitrosamine impurities.

In a further embodiment of E18, the drug product has less than 0.03 ppm nitrosamine impurities.

In yet another embodiment of E18, the drug product has less than 0.003 ppm nitrosamine impurities.

In a further embodiment E19, the invention provides a method for reducing nicotine addiction or aiding in the cessation or lessening of tobacco use in a mammal, comprising administering to said mammal an amount of a compound according to any one embodiments E1-E13 that is effective in reducing nicotine addiction or aiding in the cessation of lessening of tobacco use.

In another aspect of the invention of embodiments E1 to E19, a drug product containing a prodrug as described herein is nitrosamine free.

In another aspect of the invention of embodiments E1 to E19, a drug product containing a prodrug as described herein contains an acceptable intake (AI) limit for nitrosamine impurities of less than 96 ng/day. In certain embodiments, the acceptable intake (AI) limit for nitrosamine impurities is less than 26.5 ng/day.

The AI limit is a daily exposure to a compound such as NDMA, NDEA, NMBA, NMPA, NIPEA, or NDIPA that approximates a 1:100,000 cancer risk after 70 years of exposure. The conversion of AI limit into ppm varies by product and is calculated based on a drug's maximum daily dose (MDD) as reflected in the drug label (ppm=AI (ng)/MDD (mg)).

### Measurement of Levels of Nitrosamine in Drug Product

There are several methods that can be used to measure the level of nitrosamines in drug product. Some are specific for individual impurities.

One method using liquid chromatography-high resolution mass spectrometry (LC-HRMS) can be used for detecting level of nitrosamines in a drug product.

Another method is liquid chromatography-tandem mass spectrometry (LC-MS/MS).

Alternatively, levels of impurity in drug product can be measured more directly using ion chromatography.

Additional information of these various methods can be found in the FDA: Control of Nitrosamine Impurities in Human Drugs, Guidance for Industry (February 2021).

The compounds of the present invention may contain asymmetric or chiral centers, and, therefore, exist in different stereoisomeric forms. Unless specified otherwise, it is intended that all stereoisomeric forms of the compounds of the present invention as well as mixtures thereof, including racemic mixtures, form part of the present invention. In addition, the present invention embraces all geometric and positional isomers. For example, if a compound of the present invention incorporates a double bond or a fused ring, both the cis- and trans- forms, as well as mixtures, are embraced within the scope of the invention.

Chiral compounds of the invention (and chiral precursors thereof) may be obtained in enantiomerically-enriched form using chromatography, typically high pressure liquid chromatography (HPLC) or supercritical fluid chromatography (SFC), on a resin with an asymmetric stationary phase and with a mobile phase consisting of a hydrocarbon, typically heptane or hexane, containing from 0 to 50% isopropanol, typically from 2 to 20%, and from 0 to 5% of an alkylamine, typically 0.1% diethylamine (DEA) or isopropylamine. Concentration of the eluent affords the enriched mixture. In the case where SFC is used, the mobile phase may consist of a supercritical fluid, typically carbon dioxide, containing 2-50% of an alcohol, such as methanol, ethanol or isopropanol.

Diastereomeric mixtures can be separated into their individual diastereoisomers on the basis of their physical chemical differences by methods well known to those skilled in the art, such as by chromatography and/or fractional crystallization. Enantiomers can be separated by converting the enantiomeric mixture into a diastereomeric mixture by reaction with an appropriate optically active compound (e.g. chiral auxiliary such as a chiral alcohol or Mosher's acid chloride), separating the diastereoisomers and converting (e.g. hydrolyzing) the individual diastereoisomers to the corresponding pure enantiomers. Enantiomers can also be separated by use of a chiral HPLC column. Alternatively, the specific stereoisomers may be synthesized by using an optically active starting material, by asymmetric synthesis using optically active reagents, substrates, catalysts or solvents, or by converting one stereoisomer into the other by asymmetric transformation.

Where the compounds of the present invention possess two or more stereogenic centers and the absolute or relative stereochemistry is given in the name, the designations R and S refer respectively to each stereogenic center in ascending numerical order (1, 2, 3, etc.) according to the conventional IUPAC number schemes for each molecule. Where the compounds of the present invention possess one or more stereogenic centers and no stereochemistry is given in the name or structure, it is understood that the name or structure is intended to encompass all forms of the compound, including the racemic form.

The compounds of this invention may contain olefin-like double bonds. When such bonds are present, the compounds of the invention exist as cis and trans configurations and as mixtures thereof. The term “cis” refers to the orientation of two substituents with reference to each other and the plane of the ring (either both “up” or both “down”). Analogously, the term “trans” refers to the orientation of two substituents with reference to each other and the plane of the ring (the substituents being on opposite sides of the ring).

It is also possible that the intermediates and compounds of the present invention may exist in different tautomeric forms, and all such forms are embraced within the scope of the invention. The term “tautomer” or “tautomeric form” refers to structural isomers of different energies which are interconvertible via a low energy barrier. For example, proton tautomers (also known as prototropic tautomers) include interconversions via migration of a proton, such as keto-enol and imine-enamine isomerizations.

Valence tautomers include interconversions by reorganization of some of the bonding electrons.

Included within the scope of the claimed compounds present invention are all stereoisomers, geometric isomers and tautomeric forms of the compounds of The present invention, including compounds exhibiting more than one type of isomerism, and mixtures of one or more thereof

The present invention includes all pharmaceutically acceptable isotopically-labelled compounds of The present invention wherein one or more atoms are replaced by atoms having the same atomic number, but an atomic mass or mass number different from the atomic mass or mass number usually found in nature.

Examples of isotopes suitable for inclusion in the compounds of the invention include isotopes of hydrogen, such as ²H and ³H, carbon, such as ¹¹C, ¹³C and ¹⁴C, chlorine, such as ³⁶Cl, fluorine, such as ¹⁸F, iodine, such as ¹²³I, ¹²⁴I and ¹²⁵I, nitrogen, such as ¹³N and ¹⁵N, oxygen, such as ¹⁵O, ¹⁷O and ¹⁸O, phosphorus, such as ³²P, and sulphur, such as ³⁵S.

Certain isotopically-labelled compounds of The present invention, for example, those incorporating a radioactive isotope, are useful in drug and/or substrate tissue distribution studies. The radioactive isotopes tritium, i.e. ³H, and carbon-14, i.e. ¹⁴C, are particularly useful for this purpose in view of their ease of incorporation and ready means of detection.

Substitution with heavier isotopes such as deuterium, i.e. ²H, may afford certain therapeutic advantages resulting from greater metabolic stability, for example, increased in vivo half-life or reduced dosage requirements, and hence may be preferred in some circumstances.

Substitution with positron emitting isotopes, such as C, ¹⁸F, ¹⁵O and ¹³N, can be useful in Positron Emission Tomography (PET) studies for examining substrate receptor occupancy.

Isotopically-labelled compounds of The present invention can generally be prepared by conventional techniques known to those skilled in the art or by processes analogous to those described in the accompanying Examples and Preparations using an appropriate isotopically-labelled reagent in place of the non-labelled reagent previously employed.

The compounds of the present invention may be isolated and used per se, or when possible, in the form of its pharmaceutically acceptable salt. The term “salts” refers to inorganic and organic salts of a compound of the present invention. These salts can be prepared in situ during the final isolation and purification of a compound, or by separately treating the compound with a suitable organic or inorganic acid and isolating the salt thus formed.

Salts encompassed within the term “pharmaceutically acceptable salts” refer to the compounds of this invention which are generally prepared by reacting the free base with a suitable organic or inorganic acid to provide a salt of the compound of the invention that is suitable for administration to a patient. Suitable acid addition salts are formed from acids which form non-toxic salts. Examples include the acetate, adipate, aspartate, benzoate, besylate, bicarbonate/carbonate, bisulphate/sulphate, borate, camsylate, citrate, cyclamate, edisylate, esylate, formate, fumarate, gluceptate, gluconate, glucuronate, hexafluorophosphate, hibenzate, hydrochloride/chloride, hydrobromide/bromide, hydroiodide/iodide, isethionate, lactate, malate, maleate, malonate, mesylate, methylsulphate, naphthylate, 2-napsylate, nicotinate, nitrate, orotate, oxalate, palmitate, pamoate, phosphate/hydrogen phosphate/dihydrogen phosphate, pyroglutamate, saccharate, stearate, succinate, tannate, tartrate, tosylate, trifluoroacetate and xinofoate salts. See e.g. Berge, et al. J. Pharm. Sci. 66, 1-19 (1977); Handbook of Pharmaceutical Salts: Properties, Selection, and Use by Stahl and Wermuth (Wiley-VCH, 2002).

The compounds of the present invention, or a pharmaceutically acceptable salt thereof, may exist in unsolvated and solvated forms. The term ‘solvate’ is used herein to describe a molecular complex comprising the compound of The present invention, or a pharmaceutically acceptable salt thereof, and one or more pharmaceutically acceptable solvent molecules, for example, ethanol. The term ‘hydrate’ is employed when said solvent is water.

A currently accepted classification system for organic hydrates is one that defines isolated site, channel, or metal-ion coordinated hydrates—see Polymorphism in Pharmaceutical Solids by K. R. Morris (Ed. H. G. Brittain, Marcel Dekker, 1995). Isolated site hydrates are ones in which the water molecules are isolated from direct contact with each other by intervening organic molecules. In channel hydrates, the water molecules lie in lattice channels where they are next to other water molecules. In metal-ion coordinated hydrates, the water molecules are bonded to the metal ion.

When the solvent or water is tightly bound, the complex may have a well-defined stoichiometry independent of humidity. When, however, the solvent or water is weakly bound, as in channel solvates and hygroscopic compounds, the water/solvent content may be dependent on humidity and drying conditions. In such cases, non-stoichiometry will be the norm.

Also included within the scope of the invention are multi-component complexes (other than salts and solvates) wherein the drug and at least one other component are present in stoichiometric or non-stoichiometric amounts. Complexes of this type include clathrates (drug-host inclusion complexes) and co-crystals. The latter are typically defined as crystalline complexes of neutral molecular constituents which are bound together through non-covalent interactions, but could also be a complex of a neutral molecule with a salt. Co-crystals may be prepared by melt crystallization, by recrystallization from solvents, or by physically grinding the components together—see Chem Commun, 17, 1889-1896, by O. Almarsson and M. J. Zaworotko (2004). For a general review of multi-component complexes, see J Pharm Sci, 64 (8), 1269-1288, by Haleblian (August 1975).

The compounds of the invention include compounds as hereinbefore defined, polymorphs, and isomers thereof (including optical, geometric and tautomeric isomers) as hereinafter defined and isotopically labelled compounds of the present invention. A compound of the invention may be administered in the form of a prodrug. Thus, certain derivatives of a compound of the invention which may have little or no pharmacological activity themselves may, when administered into or onto the body, be converted into a compound of the invention having the desired activity, for example by hydrolytic cleavage, particularly hydrolytic cleavage promoted by an esterase or peptidase enzyme. Such derivatives are referred to as ‘prodrugs’. Further information on the use of prodrugs may be found in ‘The Expanding Role of Prodrugs in Contemporary Drug Design and Development, Nature Reviews Drug Discovery, 17, 559-587 (2018) (J. Rautio et al.).

Prodrugs in accordance with the invention may, for example, be produced by replacing appropriate functionalities present in compounds of the invention with certain moieties known to those skilled in the art as ‘pro-moieties’ as described, for example, in ‘Design of Prodrugs’ by H. Bundgaard (Elsevier, 1985).

Thus, a prodrug in accordance with the invention may be (a) an ester or amide derivative of a carboxylic acid when present in a compound of the invention; (b) an ester, carbonate, carbamate, phosphate or ether derivative of a hydroxyl group when present in a compound of the invention; (c) an amide, imine, carbamate or amine derivative of an amino group when present in a compound of the invention; (d) a thioester, thiocarbonate, thiocarbamate or sulfide derivatives of a thiol group when present in a compound of the invention; or (e) an oxime or imine derivative of a carbonyl group when present in a compound of the invention.

Some specific examples of prodrugs in accordance with the invention include:
(i) when a compound of the invention contains a carboxylic acid functionality (—COOH), an ester thereof, such as a compound wherein the hydrogen of the carboxylic acid functionality of the compound is replaced by C₁-C₈alkyl (e.g., ethyl) or (C₁-C₈alkyl)C(═O)OCH₂— (e.g., ^{t}BuC(═O)OCH₂—);
(ii) when a compound of the invention contains an alcohol functionality (—OH), an ester thereof, such as a compound wherein the hydrogen of the alcohol functionality of the compound is replaced by —CO(C₁-C₈ alkyl) (e.g., methylcarbonyl) or the alcohol is esterified with an amino acid;
(iii) when a compound of the invention contains an alcohol functionality (—OH), an ether thereof, such as a compound wherein the hydrogen of the alcohol functionality of the compound is replaced by (C₁-C₈ alkyl)C(═O)OCH₂— or —CH₂OP(═O)(OH)₂;
(iv) when a compound of the invention contains an alcohol functionality (—OH), a phosphate thereof, such as a compound wherein the hydrogen of the alcohol functionality of the compound is replaced by —P(═O)(OH)₂ or —P(═O)(ONa)₂ or —P(═O)(O⁻)₂Ca²⁺;
(v) when a compound of the invention contains a primary or secondary amino functionality (—NH₂ or —NHR where R≠H), an amide thereof, for example, a compound wherein, as the case may be, one or both hydrogens of the amino functionality of the compound is/are replaced by (C₁-C₁₀)alkanoyl, —COCH₂NH₂ or the amino group is derivatized with an amino acid;
(vi) when a compound of the invention contains a primary or secondary amino functionality (—NH₂ or —NHR where R≠H), an amine thereof, for example, a compound wherein, as the case may be, one or both hydrogens of the amino functionality of the compound is/are replaced by —CH₂OP(═O)(OH)₂.
(vii) when a compound of the invention contains a secondary, tertiary or quaternary amino functionality, an amine thereof, for example, a compound wherein, as the case may be, one or both hydrogens of the amino functionality of the compound is/are replaced by a carbinolamine (also hemiaminal) having the following structure:

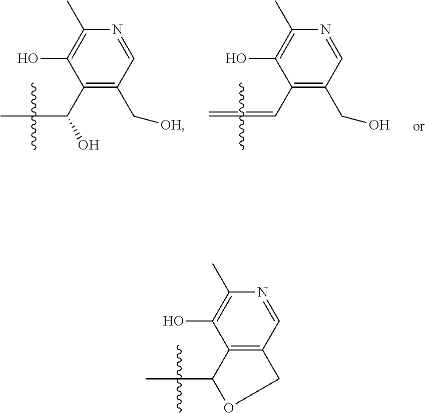 [SMILES: *[C@@H](O)c1c(CO)cnc(C)c1O ; *=Cc1c(CO)cnc(C)c1O ; "or" ; *C1OCc2cnc(C)c(O)c21]

Certain compounds of the invention may themselves act as prodrugs of other compounds the invention It is also possible for two compounds of the invention to be joined together in the form of a prodrug. In certain circumstances, a prodrug of a compound of the invention may be created by internally linking two functional groups in a compound of the invention, for instance by forming a lactone.

Certain compounds of the present invention may exist in more than one crystal form (generally referred to as “polymorphs”). Polymorphs may be prepared by crystallization under various conditions, for example, using different solvents or different solvent mixtures for recrystallization; crystallization at different temperatures; and/or various modes of cooling, ranging from very fast to very slow cooling during crystallization. Polymorphs may also be obtained by heating or melting the compound of the present invention followed by gradual or fast cooling. The presence of polymorphs may be determined by solid probe NMR spectroscopy, IR spectroscopy, differential scanning calorimetry, powder X-ray diffraction or such other techniques.

A compound of the present invention is preferably administered in the form of a pharmaceutical composition, preferably a solid oral dosage form.

The compounds of the invention can be administered alone but will generally be administered in an admixture with one or more suitable pharmaceutical excipients, adjuvants, diluents or carriers known in the art and selected with regard to the intended route of administration and standard pharmaceutical practice. The compound of the invention or combination may be formulated to provide immediate-, delayed-, modified-, sustained-, pulsed- or controlled-release dosage forms depending on the desired route of administration and the specificity of release profile, commensurate with therapeutic needs.

The pharmaceutical composition comprises a compound of the invention in an amount generally in the range of from about 1% to about 75%, 80%, 85%, 90% or even 95% (by weight) of the composition, usually in the range of about 1%, 2% or 3% to about 50%, 60% or 70%, more frequently in the range of about 1%, 2% or 3% to less than 50% such as about 25%, 30% or 35%.

Methods of preparing various pharmaceutical compositions with a specific amount of active compound are known to those skilled in this art. For examples, see Remington: The Practice of Pharmacy, Lippincott Williams and Wilkins, Baltimore Md. 20.sup.th ed. 2000.

Solid dosage forms for oral administration include capsules, tablets, chews, lozenges, pills, powders, and multi-particulate preparations (granules). In such solid dosage forms, a compound of the present invention or a combination is admixed with at least one inert excipient, diluent or carrier. Suitable excipients, diluents or carriers include materials such as sodium citrate or dicalcium phosphate and/or (a) one or more fillers or extenders (e.g., microcrystalline cellulose (available as Avicel™ from FMC Corp.) starches, lactose, sucrose, mannitol, silicic acid, xylitol, sorbitol, dextrose, calcium hydrogen phosphate, dextrin, alpha-cyclodextrin, beta-cyclodextrin, polyethylene glycol, medium chain fatty acids, titanium oxide, magnesium oxide, aluminum oxide and the like); (b) one or more binders (e.g., carboxymethylcellulose, methylcellulose, hydroxypropylcellulose, hydroxypropylmethylcellulose, gelatin, gum arabic, ethyl cellulose, polyvinyl alcohol, pullulan, pregelatinized starch, agar, tragacanth, alginates, gelatin, polyvinylpyrrolidone, sucrose, acacia and the like); (c) one or more humectants (e.g., glycerol and the like); (d) one or more disintegrating agents (e.g., agar-agar, calcium carbonate, potato or tapioca starch, alginic acid, certain complex silicates, sodium carbonate, sodium lauryl sulphate, sodium starch glycolate (available as Explotab™ from Edward Mendell Co.), cross-linked polyvinyl pyrrolidone, croscarmellose sodium A-type (available as Ac-di-sol™), polyacrilin potassium (an ion exchange resin) and the like); (e) one or more solution retarders (e.g., paraffin and the like); (f) one or more absorption accelerators (e.g., quaternary ammonium compounds and the like); (g) one or more wetting agents (e.g., cetyl alcohol, glycerol monostearate and the like); (h) one or more adsorbents (e.g., kaolin, bentonite and the like); and/or lone or more lubricants (e.g., talc, calcium stearate, magnesium stearate, stearic acid, polyoxyl stearate, cetanol, talc, hydrogenated caster oil, sucrose esters of fatty acid, dimethylpolysiloxane, microcrystalline wax, yellow beeswax, white beeswax, solid polyethylene glycols, sodium lauryl sulfate and the like). In the case of capsules and tablets, the dosage forms may also comprise buffering agents.

Solid compositions of a similar type may also be used as fillers in soft or hard filled gelatin capsules using such excipients as lactose or milk sugar, as well as high molecular weight polyethylene glycols, and the like.

Solid dosage forms such as tablets, dragees, capsules, and granules may be prepared with coatings and shells, such as enteric coatings and others well known in the art. They may also contain opacifying agents and can also be of such composition that they release the compound of the present invention and/or the additional pharmaceutical agent in a delayed manner. Examples of embedding compositions that can be used are polymeric substances and waxes. The drug may also be in micro-encapsulated form, if appropriate, with one or more of the above-mentioned excipients.

For tablets, the active agent will typically comprise less than 50% (by weight) of the formulation, for example less than about 10% such as 5% or 2.5% by weight. The predominant portion of the formulation comprises fillers, diluents, disintegrants, lubricants and optionally, flavors. The composition of these excipients is well known in the art. Frequently, the fillers/diluents will comprise mixtures of two or more of the following components: microcrystalline cellulose, mannitol, lactose (all types), starch, and di-calcium phosphate. The filler/diluent mixtures typically comprise less than 98% of the formulation and preferably less than 95%, for example 93.5%. Preferred disintegrants include Ac-di-sol™, Explotab™, starch and sodium lauryl sulphate. When present a disintegrant will usually comprise less than 10% of the formulation or less than 5%, for example about 3%. A preferred lubricant is magnesium stearate. When present a lubricant will usually comprise less than 5% of the formulation or less than 3%, for example about 1%.

Tablets may be manufactured by standard tabletting processes, for example, direct compression or a wet, dry or melt granulation, melt congealing process and extrusion. The tablet cores may be mono or multi-layer(s) and can be coated with appropriate overcoats known in the art.

A dosage of the compound of the present invention is used that is effective for the indication being treated. Such dosages can be determined by standard assays such as those referenced above and provided herein.

These dosages are based on an average human subject having a weight of about 60 kg to 70 kg. The physician will readily be able to determine doses for subjects whose weight falls outside this range, such as infants and the elderly.

Dosage regimens may be adjusted to provide the optimum desired response. For example, a single loading dose may be administered, several divided doses may be administered over time or the dose may be proportionally reduced or increased as indicated by the exigencies of the therapeutic situation. Dosage unit form, as used herein, refers to physically discrete units suited as unitary dosages for the mammalian subjects to be treated; each unit containing a predetermined quantity of active compound calculated to produce the desired therapeutic effect in association with the required pharmaceutical carrier. The specification for the dosage unit forms of the invention are dictated by and directly dependent on (a) the unique characteristics of the chemotherapeutic agent and the particular therapeutic or prophylactic effect to be achieved, and (b) the limitations inherent in the art of compounding such an active compound for the treatment of sensitivity in individuals.

Thus, the skilled artisan would appreciate, based upon the disclosure provided herein, that the dose and dosing regimen is adjusted in accordance with methods well-known in the therapeutic arts. That is, the maximum tolerable dose can be readily established, and the effective amount providing a detectable therapeutic benefit to a patient may also be determined, as can the temporal requirements for administering each agent to provide a detectable therapeutic benefit to the patient. Accordingly, while certain dose and administration regimens are exemplified herein, these examples in no way limit the dose and administration regimen that may be provided to a patient in practicing the present invention.

It is to be noted that dosage values may vary with the type and severity of the condition to be alleviated and may include single or multiple doses. It is to be further understood that for any particular subject, specific dosage regimens should be adjusted over time according to the individual need and the professional judgment of the person administering or supervising the administration of the compositions, and that dosage ranges set forth herein are exemplary only and are not intended to limit the scope or practice of the claimed composition. For example, doses may be adjusted based on pharmacokinetic or pharmacodynamic parameters, which may include clinical effects such as toxic effects and/or laboratory values. Thus, the present invention encompasses intra-patient dose-escalation as determined by the skilled artisan. Determining appropriate dosages and regiments for administration of the chemotherapeutic agent are well-known in the relevant art and would be understood to be encompassed by the skilled artisan once provided the teachings disclosed herein.

A pharmaceutical composition of the invention may be prepared, packaged, or sold in bulk, as a single unit dose, or as a plurality of single unit doses. As used herein, a “unit dose” is discrete amount of the pharmaceutical composition comprising a predetermined amount of the active ingredient. The amount of the active ingredient is generally equal to the dosage of the active ingredient which would be administered to a subject or a convenient fraction of such a dosage such as, for example, one-half or one-third of such a dosage.

These agents and compounds of the invention can be combined with pharmaceutically acceptable carriers. The particular dosage regimen, i.e., dose, timing and repetition, will depend on the particular individual and that individual's medical history.

Acceptable carriers, excipients, or stabilizers are nontoxic to recipients at the dosages and concentrations employed, and may comprise buffers such as phosphate, citrate, and other organic acids; salts such as sodium chloride; antioxidants including ascorbic acid and methionine; preservatives (such as octadecyldimethylbenzyl ammonium chloride; hexamethonium chloride; benzalkonium chloride, benzethonium chloride; phenol, butyl or benzyl alcohol; alkyl parabens, such as methyl or propyl paraben; catechol; resorcinol; cyclohexanol; 3-pentanol; and m-cresol); low molecular weight (less than about 10 residues) polypeptides; proteins, such as serum albumin, gelatin, or Igs; hydrophilic polymers such as polyvinylpyrrolidone; amino acids such as glycine, glutamine, asparagine, histidine, arginine, or lysine; monosaccharides, disaccharides, and other carbohydrates including glucose, mannose, or dextrins; chelating agents such as EDTA; sugars such as sucrose, mannitol, trehalose or sorbitol; salt-forming counter-ions such as sodium; metal complexes (e.g., Zn-protein complexes); and/or non-ionic surfactants such as TWEEN™, PLURONICS™ or polyethylene glycol (PEG).

Sustained-release preparations may be used. Suitable examples of sustained-release preparations include semi-permeable matrices of solid hydrophobic polymers containing the compound of the invention, which matrices are in the form of shaped articles, e.g., films, or microcapsules.

Methods of preparing various pharmaceutical compositions with a certain amount of active ingredient are known, or will be apparent in light of this disclosure, to those skilled in this art. For examples of methods of preparing pharmaceutical compositions see Remington's Pharmaceutical Sciences, 20th Edition (Lippincott Williams & Wilkins, 2000).

Pharmaceutical compositions according to the invention may contain 0.1%-95% of the compound(s) of this invention, preferably 1%-70%. In any event, the composition to be administered will contain a quantity of a compound(s) according to the invention in an amount effective to treat the disease/condition of the subject being treated.

### EXPERIMENTALS

The following illustrate the synthesis of various compounds of the present invention. Additional compounds within the scope of this invention may be prepared using the methods illustrated in these Examples, either alone or in combination with techniques generally known in the art. All starting materials in these Preparations and Examples are either commercially available or can be prepared by methods known in the art or as described herein.

### Example 1

U.S. Pat. No. 6,410,550, the disclosure of which is hereby incorporated by reference in its entirety, describes aryl-fuse azapolycyclic compounds, methods for synthesis and their use in various disease states including reducing nicotine addiction or aiding in the cessation or lessening of tobacco use. Example 26 of the '550 patent is specific to the compound 5,8,14-triazatetracyclo[10.3.1.0^{2,11}.0^{4,9}]hexadeca-2(11),3,5,7,9-pentaene hydrochloride (varenicline)

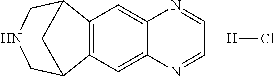

that is synthesized by the following steps:

### A) 1-(4,5-Diamino-10-aza-tricyclo[6.3.1.0^{2,7}]dodeca-2(7),3,5-trien-10-yl)-2,2,2-trifluoro-ethanone

1-(4,5-Dinitro-10-aza-tricyclo[6.3.1.0^{2,7}]dodeca-2(7),3,5-trien-10-yl)-2,2,2-trifluoro-ethanone (3.0 g, 8.70 mmol) was hydrogenated in MeOH (30 ml) under H₂ (45 psi) over Pd(OH)₂ (300 mg of 20 wt %/C, 10% wt). After 2.5 hours the reaction was filtered through a Celite pad and rinsed with MeOH (30 ml). The solution was concentrated to a light brown oil which crystallized (2.42 g, 96%). (TLC 10% MeOH/CH₂Cl₂ R_{f} 0.56). APCI MS m/e 286.2 [(M+1)⁺]. mp 129-131 °C.

### B) 1-(5,8,14-Triazatetracyclo[10.3.1.0^{2,11}.0^{4,9}]hexadeca-2(11),3,5,7,9-pentaene)-2,2,2-trifluoro-ethanone

1-(4,5-Diamino-10-aza-tricyclo[6.3.1.0^{2,7}]dodeca-2(7),3,5-trien-10-yl)-2,2,2-trifluoro-ethanone (500 mg, 1.75 mmol) was stirred in THF (2 ml). This mixture was treated with H₂O (2 mL) and glyoxal sodium bisulfite addition compound hydrate (931 mg, 3.50 mmol) then stirred at 55 °C for 2.5 hours. The reaction was cooled to room temperature and extracted with EtOAc (3×40 ml). The combined organic layer was washed with H₂O (2×30 ml), dried (Na₂SO₄), filtered, concentrated and chromatographed on Silica gel to provide an off white powder (329 mg, 60%). (TLC 25% EtOAc/hexanes R_{f} 0.40). mp 164-166 °C.

### C) 5,8,14-Triazatetracyclo[10.3.1.0^{2,11}.0^{4,9}]hexadeca-2(11),3,5,7,9-pentaene hydrochloride

1-(5,8,14-Triazatetracyclo[10.3.1.0^{2,11}.0^{4,9}]hexadeca-2(11),3,5,7,9-pentaene)-2,2,2-trifluoro-ethanone (320 mg, 1.04 mmol) was slurried in MeOH (2.0 ml) and treated with Na₂CO₃ (221 mg, 2.08 mmol) in H₂O (2.0 ml). The mixture was warmed to 70 °C for 2 hours, then concentrated, treated with H₂O (20 mL) and extracted with CH₂Cl₂ (3×10 ml). The organic layer was dried through a cotton plug and concentrated to give a light yellow oil (183 mg, 83%) which solidified upon standing (mp 138-140 °C). This material was dissolved in MeOH (10 mL), treated with 3M HCl/EtOAc (3 ml), concentrated and azeotroped with MeOH (2×20 mL) to give solids which were recrystallized from MeOH/Et₂O to afford product as a white solid (208 mg, 97%). (TLC 5% MeOH/CH₂Cl₂ (NH₃) R_{f} 0.26). ¹H NMR (400 MHz, CD₃OD) δ8.94 (s, 2H), 8.12 (s, 2H), 3.70 (m, 2H), 3.54 (d, J=12.5 Hz, 2H), 3.35 (d, J=12.5 Hz, 2H), 2.49 (m, 1H), 2.08 (d, J=11.0 Hz, 1H). GCMS m/e 211 (M+). mp 225-230 °C.

### Example 2

U.S. Pat. Nos. 6,890,927 and 7,265,119, the disclosures of which is hereby incorporated by reference in its entirety, describe polymorphic and salt forms of 5,8,14-triazatetracyclo [10.3.1.0^{2,11}.0^{4,9}]hexadeca-2(11),3,5,7,9-pentaene (varenicline), including the commercial tartrate salt form that was approved by the U.S. FDA and various other world regulatory agencies as an aid to smoking cessation treatment.

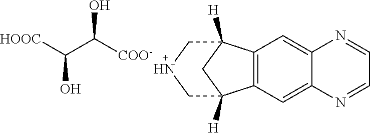

### Example 3

### (R)-6-methyl-1-((6R,10S)-6,7,9,10-tetrahydro-8H-6,10-methanoazepino[4,5-g]quinoxalin-8-yl)-1,3-dihydrofuro[3,4-c]pyridin-7-ol (Compound 1)

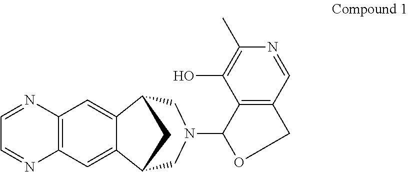 [SMILES: Cc1ncc2c(c1O)C(N1C[C@H]3C[C@@H](C1)c1cc4nccnc4cc13)OC2]

To a 200 mL Easymax reactor at ambient temperature was added, Varenicline Tartrate (10.0 g, 27.12 mmol) and Pyridoxal Hydrochloride (1 equiv., 27.12 mmol, 5.577 g). Followed by THF (200 mL) and Water (25 mL). The resulting mixture is stirred into a biphasic light slurry. Na₂CO₃ (3 equiv., 81.35 mmol, 8.622 g) was dissolved into Water (25 mL) to give a Na₂CO₃ solution, this was added dropwise to the reaction mixture while controlling the CO₂ off gas with the reaction temperature kept below 25 °C. After the addition, pH was checked to be ~10. The resulting biphasic mixture was stirred for 30 min. Sodium Sulfate (Anhydrous) (1.50 g) was added. The resulting mixture was stirred for 1 h. The bottom phase clearly had solid presence indicating a saturated aqueous phase. The phases were separated, and the aqueous phase was extracted with THF (100 mL). The organic phases were combined, dried over anhydrous Magnesium Sulfate (2 g) and filtered. The filter cake was rinsed with THF (20 mL). The resulting filtrate was concentrated to a low stirrable volume under atmospheric pressure, then solvent-exchanged with EtOAc to remove most THF. The final volume was ~30 mL. The resulting slurry was cooled to 20 °C ramping at 1 k/min, then granulated for 2 h and filtered. 9.16 g product isolated as an off white powder.

1H NMR (400 MHz CDCl₃) δ 8.83-9.79 (m, 2H), 7.92 (s, 1H), 7.91 (s, 1H), 7.81 (s, 1H), 5.72-5.68 (m br, 1H), 5.10-4.92 (m, 3H), 3.58-3.52 (m, br, 1H), 3.46 (d, J=10.2 Hz, 1H), 3.42-3.37 (m br, 1H), 3.23-3.15 (m, 1H), 2.96 (d, J=10.9 Hz, 1H), 2.55-2.46 (m, 1H), 2.44-2.35 (m, 1H), 3.21 (s, 3H), 1.97 (d, J=11.0 Hz, 1H).

### Solid-State Characterization of Compound 1

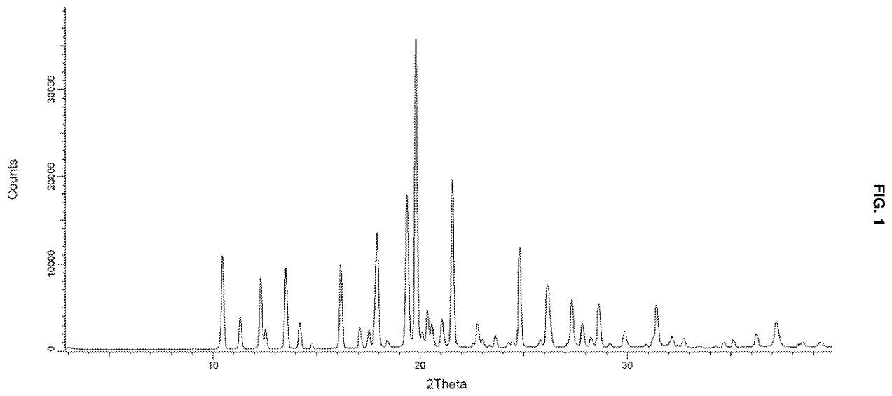

Compound 1 is a crystalline anhydrous solid form as represented by its PXRD pattern (FIG. 1). Compound 1 demonstrated less than 0.1% of weight loss up to 150 °C, that is consistent with the anhydrous nature of the solid form. Compound 1 has a melting onset of ~227 °C and is non-hygroscopic, i.e., less than 0.2% moisture uptake at 90% RH/25 °C.

### Single Crystal Structure Results

Good quality single crystals (prismatic in shape) of Compound 1 were grown from dichloromethane. Data collection was performed on a Bruker D8 Quest diffractometer at 298 K. Data collection consisted of omega and phi scans. The structure was solved by intrinsic phasing using SHELX software suite in the monoclinic space group P2₁/n. The structure was subsequently refined by the full-matrix least squares method. All non-hydrogen atoms were found and refined using anisotropic displacement parameters. The hydrogen atoms located on oxygen were found from the Fourier difference map and refined with distances restrained. The remaining hydrogen atoms were placed in calculated positions and allowed to ride on their carrier atoms. The final refinement included isotropic displacement parameters for all hydrogen atoms. The CheckCIF report does not include any “A” or “B” level alerts. The final R-index was 3.85%. A final difference Fourier revealed no missing or misplaced electron density. Pertinent crystal, data collection and refinement are summarized in Table 1.

TABLE 1
Crystal data and structure refinement for Compound 1

| Identification code | Compound 1 |
|---|---|
| Empirical formula | C₂₁H₂₀N₄O₂ |
| Formula weight | 360.41 |
| Temperature/K | 296.15 |
| Crystal system | monoclinic |
| Space group | P2₁/n |
| a/Å | 10.2166(2) |
| b/Å | 10.3494(2) |
| c/Å | 17.3213(4) |
| α/° | 90 |
| β/° | 103.6300(10) |
| γ/° | 90 |
| Volume/Å³ | 1779.90(6) |
| Z | 4 |
| ρ_{calc}g/cm³ | 1.345 |
| Radiation | CuKα (λ = 1.54178) |
| Goodness-of-fit on F² | 1.051 |
| Final R indexes [I >= 2σ (I)] | R₁ = 0.0385, wR₂ = 0.1017 |
| Final R indexes [all data] | R₁ = 0.0400, wR₂ = 0.1031 |

Table 2 provides the Powder X-ray diffraction peak list of Compound 1. Powder X-ray diffraction analysis was conducted using a Bruker AXS D8 Endeavor diffractometer equipped with a Cu radiation source (CuKα̅ λ=1.5418 Å). The divergence slit was set at 10 mm continuous illumination. Diffracted radiation was detected by a PSD-Lynx Eye detector, with the detector PSD opening set at 2.99 degrees. The X-ray tube voltage and amperage were set to 40 kV and 40 mA respectively. Data was collected in the Theta-Theta goniometer at the Cu wavelength from 3.0 to 40.0 degrees 2-Theta using a step size of 0.02 degrees and a step time of 0.3 second. The antiscatter screen was set to a fixed distance of 1.5 mm. Samples were rotated at 15/min during collection. Samples were prepared by placing them in a silicon low background sample holder and rotated during collection. Data were collected using Bruker DIFFRAC Plus software and analysis was performed by EVA diffract plus software. Using the peak search algorithm in the EVA software, peaks selected with a threshold value of 1 were used to make preliminary peak assignments. To ensure validity, adjustments were manually made; the output of automated assignments was visually checked and peak positions were adjusted to the peak maximum. Peaks with relative intensity of ≥3% were generally chosen. The peaks which were not resolved or were consistent with noise were not selected. A typical error associated with the peak position from PXRD stated in USP up to +/−0.2° 2-Theta (USP-941).

TABLE 2

| | Relative |
|---|---|
| 2theta (°) | Intensity (%) |
| 10.4 | 43.7 |
| 11.3 | 15.0 |
| 12.3 | 32.9 |
| 12.5 | 8.8 |
| 13.5 | 37.0 |
| 14.2 | 12.1 |
| 16.2 | 38.6 |
| 17.1 | 9.1 |
| 17.5 | 8.6 |
| 17.9 | 52.6 |
| 19.4 | 47.8 |
| 19.8 | 100.0 |
| 20.3 | 11.8 |
| 20.6 | 7.6 |
| 21.1 | 8.5 |
| 21.6 | 53.4 |
| 22.8 | 7.6 |
| 23.6 | 3.8 |
| 24.8* | 29.6 |
| 26.1 | 18.4 |
| 27.3 | 16.3 |
| 27.8 | 8.0 |
| 28.2 | 3.5 |
| 28.6 | 14.8 |
| 29.9 | 5.0 |
| 31.4 | 16.0 |
| 32.1 | 3.9 |
| 32.7 | 3.3 |
| 35.1 | 3.1 |
| 36.3 | 4.0 |
| 37.2 | 8.8 |

### Pharmacological Data

### Experiment 1

### Study to Assess the Bioequivalence of Compound 1 and Varenicline Tartrate

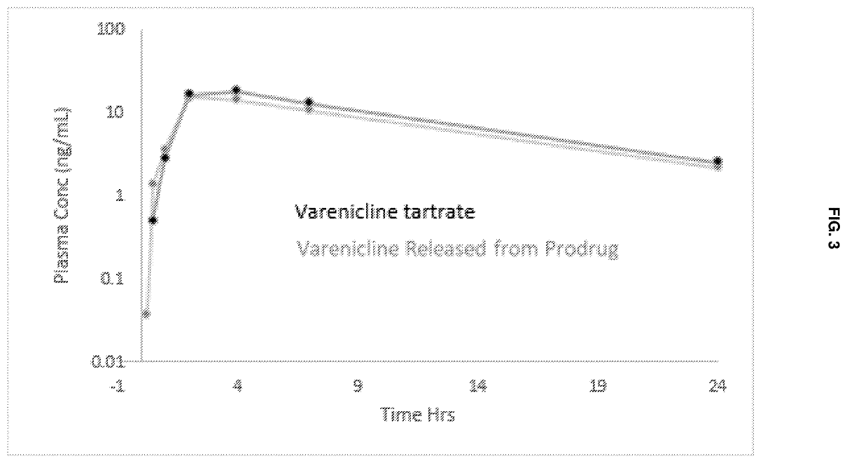

A prodrug of varenicline (Compound 1) prepared as described above in Example 1 was orally dosed in capsule form to non-human primates to assess bioequivalence to varenicline tartrate API (Chantix). FIG. 3 is a graph of the mean monkey plasma concentrations of varenicline following an oral dose of 0.04 mg eq/kg of prodrug (Compound 1) and 0.04 mg eq/kg of varenicline tartrate. The data provides evidence that the bioequivalence (plasma concentration over time) of the varenicline released from the prodrug is comparable to varenicline tartrate. The data also provides evidence that there was no prodrug remaining in the systemic circulation.

### Experiment 2

### Study to Evaluate Level of Nitrosamine Formation of Compound 1 Under Nitrosating Conditions as Compared to Varenicline Tartrate

A study was conducted to determine whether varenicline prodrug (Compound 1) forms nitrosamine under nitrosating conditions in solution as compared to varenicline. Regular UPLC-MS detection was utilized. Varenicline free base (0.1 mmol), varenicline tartrate salt (0.1 mmol), and Compound 1 (0.1 mmol) were added to separate vials. THF (2.2 ml) was added to each vial. NaNO₂ (0.1 eq) and AcOH (0.1 eq) (added as a 0.1 wt % NaNO₂ solution with equivalent molar of acetic acid was also added to each vial to provide a total volume of ~2.5 ml/vial.

The data in Table 3 shows that the nitrosamine prodrug (Compound 1) does not form nitrosamines under nitrosating conditions as compared to varenicline tartrate.

TABLE 3
Level of nitrosamine levels under nitrosating conditions over time

| | 10 min. N—NO | 2 h N—NO | 42 h N—NO |
|---|---|---|---|
| Varenicline Compound | Level | Level | Level |
| Varenicline Free base | Not detected | Not detected | Not detected |
| Varenicline Prodrug | Not detected | Not detected. | Not detected |
| (Compound 1) | | | |
| Varenicline tartrate | Not detected by UV | 0.83 A % | 5.15 A % |
| | Detectable by MS | (210 nm) | (210 nm) |

A % = area % by UV

### Varenicline Clinical Efficacy

The efficacy of varenicline in smoking cessation was demonstrated in six clinical trials in which a total of 3659 chronic cigarette smokers (≥10 cigarettes per day) were treated with varenicline (FDA Label, LAB-0327-7.0 January 2008). In all clinical studies, abstinence from smoking was determined by patient selfreport and verified by measurement of exhaled carbon monoxide (CO≤10 ppm) at weekly visits. Among the varenicline treated patients enrolled in these studies, the completion rate was 65%. Except for the initial Phase 2 study (Study 1) and the maintenance of abstinence study (Study 6), patients were treated for 12 weeks and then were followed for 40 weeks posttreatment. Most subjects enrolled in these trials were white (79% 96%). All studies enrolled almost equal numbers of men and women. The average age of subjects in these studies was 43 years. Subjects on average had smoked about 21 cigarettes per day for an average of approximately 25 years.

In all studies, patients were provided with an educational booklet on smoking cessation and received up to 10 minutes of smoking cessation counseling at each weekly treatment visit according to Agency for Healthcare Research and Quality guidelines. Patients set a date to stop smoking (target quit date, TQD) with dosing starting 1 week before this date.

Study 1: This was a six-week dose-ranging study comparing varenicline to placebo. This study provided initial evidence that varenicline at a total dose of 1 mg per day or 2 mg per day was effective as an aid to smoking cessation.

Study 2: This study of 627 subjects compared varenicline 1 mg per day and 2 mg per day with placebo. Patients were treated for 12 weeks (including one week titration) and then were followed for 40 weeks post-treatment. varenicline was given in two divided doses. Each dose of varenicline was given in two different regimens, with and without initial dose titration, to explore the effect of different dosing regimens on tolerability. For the titrated groups, dosage was titrated up over the course of one week, with full dosage achieved starting with the second week of dosing. The titrated and nontitrated groups were pooled for efficacy analysis.

Forty five percent of subjects receiving varenicline 1 mg per day (0.5 mg BID) and 51% of subjects receiving 2 mg per day (1 mg BID) had co-confirmed continuous abstinence during weeks 9 through 12 compared to 12% of subjects in the placebo group. In addition, 31% of the 1 mg per day group and 31% of the 2 mg per day group were continuously abstinent from one week after TQD through the end of treatment as compared to 8% of the placebo group.

Study 3: This flexible-dosing study of 312 subjects examined the effect of a patient-directed dosing strategy of varenicline or placebo. After an initial one-week titration to a dose of 0.5 mg BID, subjects could adjust their dosage as often as they wished between 0.5 mg QD to 1 mg BID per day. Sixty nine percent of patients titrated to the maximum allowable dose at any time during the study. For 44% of patients, the modal dose selected was 1 mg BID; for slightly over half of the study participants, the modal dose selected was 1 mg/day or less.

Of the subjects treated with varenicline, 40% had co-confirmed continuous abstinence during weeks 9 through 12 compared to 12% in the placebo group. In addition, 29% of the VARENICLINE group were continuously abstinent from one week after TQD through the end of treatment as compared to 9% of the placebo group.

Study 4 and Study 5: These identical double-blind studies compared varenicline 2 mg per day, bupropion sustained release (SR) 150 mg BID, and placebo. Patients were treated for 12 weeks and then were followed for 40 weeks post-treatment. The varenicline dosage of 1 mg BID was achieved using a titration of 0.5 mg QD for the initial 3 days followed by 0.5 mg BID for the next 4 days. The bupropion SR dosage of 150 mg BID was achieved using a 3-day titration of 150 mg QD. Study 4 enrolled 1022 subjects and Study 5 enrolled 1023 subjects. Patients inappropriate for bupropion treatment or patients who had previously used bupropion were excluded.

In Study 4, subjects treated with varenicline had a superior rate of co-confirmed abstinence during weeks 9 through 12 (44%) compared to patients treated with bupropion SR (30%) or placebo (17%). The bupropion SR quit rate was also superior to placebo. In addition, 29% of the VARENICLINE group were continuously abstinent from one week after TQD through the end of treatment as compared to 12% of the placebo group and 23% of the bupropion SR group.

Similarly in Study 5, subjects treated with varenicline had a superior rate of co-confirmed abstinence during weeks 9 through 12 (44%) compared to patients treated with bupropion SR (30%) or placebo (18%). The bupropion SR quit rate was also superior to placebo. In addition, 29% of the VARENICLINE group were continuously abstinent from one week after TQD through the end of treatment as compared to 11% of the placebo group and 21% of the bupropion SR group.

Throughout this application, various publications are referenced. The disclosures of these publications in their entireties are hereby incorporated by reference into this application for all purposes.

It will be apparent to those skilled in the art that various modifications and variations can be made in the present invention without departing from the scope or spirit of the invention. Other embodiments of the invention will be apparent to those skilled in the art from consideration of the specification and practice of the invention disclosed herein. It is intended that the specification and examples be considered as exemplary only, with a true scope and spirit of the invention being indicated by the following claims.

1. A compound that is (R)-6-methyl-1-((6R,10S)-6,7,9,10-tetrahydro-8H-6,10-methanoazepino[4,5-g]quinoxalin-8-yl)-1,3-dihydrofuro[3,4-c]pyridin-7-ol, or a pharmaceutically acceptable salt thereof, tautomer thereof, or pharmaceutically acceptable salt of a tautomer thereof.

2. The compound of claim 1, wherein the compound is formula: 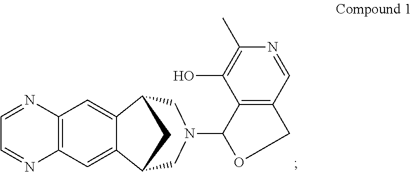 [SMILES: Cc1ncc2c(c1O)C(N1C[C@H]3C[C@@H](C1)c1cc4nccnc4cc13)OC2]
or a pharmaceutically acceptable salt thereof.

3. The compound of claim 2, wherein the compound is crystalline.

4. The compound of claim 3, wherein the crystalline form exhibits a powder X-ray diffraction pattern (PXRD) having at least one characteristic peak expressed in degrees 2θ (CuKα radiation) selected from the group consisting of 10.4±0.2° 2θ, 11.3±0.2° 2θ, 13.5±0.2° 2θ, 14.2±0.2° 2θ, 17.9±0.2° 2θ, and 24.8±0.2° 2θ.

5. The compound of claim 4, wherein the crystalline form exhibits a powder X-ray diffraction pattern (PXRD) having at least two characteristic peaks expressed in degrees 2θ (CuKα radiation) selected from the group consisting of 10.4±0.2° 2θ, 11.3±0.2° 2θ, 13.5±0.2° 2θ, 14.2±0.2° 2θ, 17.9±0.2° 2θ, and 24.8±0.2° 2θ.

6. The compound of claim 5, wherein the crystalline form exhibits a powder X-ray diffraction pattern (PXRD) having at least three characteristic peaks expressed in degrees 2θ (CuKα radiation) selected from the group consisting of 10.4±0.2° 2θ, 11.3±0.2° 2θ, 13.5±0.2° 2θ, 14.2±0.2° 2θ, 17.9±0.2° 2θ, and 24.8±0.2° 2θ.

7. The compound of claim 6, wherein the crystalline form exhibits a powder X-ray diffraction pattern (PXRD) having at least four characteristic peaks expressed in degrees 2θ (CuKα radiation) selected from the group consisting of 10.4±0.2° 2θ, 11.3±0.2° 2θ, 13.5±0.2° 2θ, 14.2±0.2° 2θ, 17.9±0.2° 2θ, and 24.8±0.2° 2θ.

8. The compound of claim 7, wherein the crystalline form exhibits a powder X-ray diffraction pattern (PXRD) having at least five characteristic peaks expressed in degrees 2θ (CuKα radiation) selected from the group consisting of 10.4±0.2° 2θ, 11.3±0.2° 2θ, 13.5±0.2° 2θ, 14.2±0.2° 2θ, 17.9±0.2° 2θ, and 24.8±0.2° 2θ.

9. The compound of claim 4, wherein the crystalline form exhibits a powder X-ray diffraction pattern (PXRD) having characteristic peaks expressed in degrees 2θ (CuKα radiation) of 10.4±0.2° 2θ, 11.3±0.2° 2θ, 13.5±0.2° 2θ, 14.2±0.2° 2θ, 17.9±0.2° 2θ, and 24.8±0.2° 2θ.

10. The compound of claim 1, wherein the compound is the tautomer, formula: 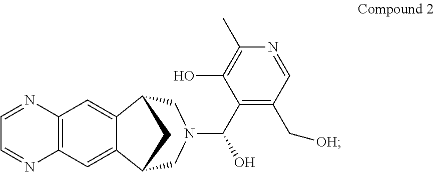 [SMILES: Cc1ncc(CO)c([C@H](O)N2C[C@H]3C[C@@H](C2)c2cc4nccnc4cc23)c1O]
or a pharmaceutically acceptable salt thereof.

11. The compound of claim 1 wherein the compound is the tautomer, formula: 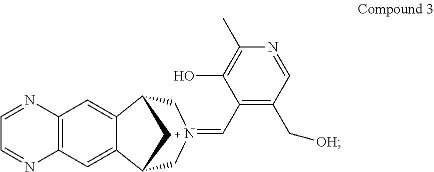
or a pharmaceutically acceptable salt thereof.

12. A composition comprising the compound according to claim 1 and at least one pharmaceutically acceptable excipient, wherein the composition is free of nitrosamine impurities.

13. A composition comprising the compound according to claim 1 and at least one pharmaceutically acceptable excipient, wherein the composition has less than 0.03 ppm nitrosamine impurities.

14. The composition of claim 13, wherein the composition has less than 0.003 ppm nitrosamine impurities.

15. A composition comprising a prodrug of a compound having a secondary, tertiary, or quaternary amine and at least one pharmaceutically acceptable excipient, wherein the composition is free of nitrosamine impurities.

16. A composition comprising a prodrug of a compound having a secondary, tertiary, or quaternary amine and at least one pharmaceutically acceptable excipient, wherein the composition has less than 0.03 ppm nitrosamine impurities.

17. The composition of claim 16, wherein the composition has less than 0.003 ppm nitrosamine impurities.

18. (canceled)

19. (canceled)

20. (canceled)

21. A method for reducing nicotine addiction or aiding in the cessation or lessening of tobacco use in a mammal, comprising administering to said mammal an amount of the compound according to claim 1 that is effective in reducing nicotine addiction or aiding in the cessation of lessening of tobacco use.
